(19) United States
(12) Patent Application Publication
(10) Pub. No.: US 2012/0095500 A1
(43) Pub. Date: Apr. 19, 2012

(54) CONCENTRIC WIRE EMBOLISM PROTECTION DEVICE

(76) Inventor: Richard R. Heuser, Phoenix, AZ (US)

(21) Appl. No.: 12/905,056

(22) Filed: Oct. 14, 2010

(51) Int. Cl. A61F 2/01

(52) U.S. Cl. 606/200

(57) ABSTRACT

An embolism protection system is disclosed that provides a first wire having a proximal end, a distal end, and an outer diameter; a second wire having a proximal end, a distal end, an inner lumen, and an outer diameter, the inner lumen sized to accommodate the outer diameter of the first wire; a third having a proximal end, a distal end, and an inner lumen, the inner lumen sized to accommodate the outer diameter of the second wire; and a substantially conical filter device formed substantially from porous metal mesh and having a central apex, a collapsed state, and an expanded state, wherein the substantially conical filter device is adapted to couple to the second wire at or near the distal end of the second wire, the second wire passing through the central apex, and wherein the substantially conical filter device is adapted to assume the expanded state when the third wire is retracted from the distal end of the second wire, and is adapted to assume the collapsed state when the third wire is advanced over the substantially conical filter device and towards the distal end of the second wire.

### CROSS REFERENCE TO RELATED APPLICATIONS

The present application is related to U.S. patent application Ser. No. 12/140,183, filed Jan. 8, 2009, entitled “CATHETER GUIDEWIRE SYSTEM USING CONCENTRIC WIRES;” and U.S. Pat. No. 7,402,141, issued Jul. 22, 2008, entitled “CATHETER GUIDEWIRE SYSTEM USING CONCENTRIC WIRES,” the disclosures of which are hereby incorporated by reference in their entirety.

### TECHNICAL FIELD

Embodiments relate to an embolism-preventing device that prevents the free flow of embolism-creating particles that are created during cardiac procedures such as percutaneous valve intervention.

### BACKGROUND

An embolus can be any particle comprising a foreign or native material that enters the vascular system with potential to cause occlusion of blood flow. Emboli can be formed from aggregated fibrin, red blood cells, collagen, cholesterol, plaque, fat, calcified plaque, bubbles, arterial tissue, and/or other miscellaneous fragments. Each dislodged fragment, or embolus, is carried along by the blood flow until it becomes lodged or trapped in a smaller vessel and occludes blood flow, creating an embolism. Since emboli reduce or cut off blood flow, damage to the body may result, such as tissue damage, heart attack, stroke, or even death.

Percutaneous valve interventions include valvuloplasty, annuloplasty, and valve replacement surgeries performed on the mitral, tricuspid, aortic, and pulmonary valves. These interventions carry a high risk of embolism formation. For instance, aortic valve applications may carry a 60-80% chance of embolism formation and subsequent cerebral ischemic events.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates a cross-sectional side view of an embodiment of the guidewire system used to deploy the embolism protection devices disclosed herein, showing three concentric wires, including the proximal and distal ends, central lumens, and proximal handles, in accordance with various embodiments;

FIG. 2 illustrates an example of an embolism protection device that has been deployed in an artery distal to a plaque, and also illustrates an example of a proximal handle for a catheter guidewire system used to deploy the embolism protection device, in accordance with various embodiments; and

FIGS. 3A-3E illustrate a first wire being inserted into an artery (FIG. 3A), the second and third wires being inserted into the same artery (FIG. 3B), retraction of the third (outer) wire to release the embolism protecting device (FIG. 3C), deployment of the embolism protecting device within the artery (FIG. 3D), and retraction of the embolism protecting device (FIG. 3E), in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments herein provide embolism protection devices that may be deployed in a body vessel for the collection of loosened or floating debris, such as embolic material dislodged during or thrombi formed as a result of percutaneous cardiac procedure, such as a valve intervention. In various embodiments, the device may be deployed in the carotid artery to protect the subject from brain embolism associated with, for instance, percutaneous treatment of the aortic, pulmonary, or mitral valve. In other embodiments, the device may be used to protect the vasculature of a patient from dislodged emboli from valvular or coronary artery disease, or during angioplasty, atherectomy, thrombectomy, embolectomy, intravascular diagnostic procedures, and/or stent placement procedures. Embodiments of the device also may be used to protect the patient from emboli resulting from open interventional procedures including transapical approaches to treat valvular disease or any minimally invasive heart intervention.

Some embodiments of the embolism protection device may be made from a fine metal mesh, such as nitinol, a metal alloy of nickel and titanium that exhibits both shape memory and superelasticity. Such mesh devices may be configured to open once deployed to a desired site, and to form a barrier across the lumen of a vessel without causing trauma to the vessel or other structures. In some embodiments, the fine metal mesh may be sufficiently porous to allow blood to pass through and prevent ischemia, while still retaining particles, such as aggregated fibrin, red blood cells, collagen, cholesterol, plaque, fat, calcified plaque, bubbles, arterial tissue, any of which may cause an embolism. Such fine metal mesh may have a pore size of between about 50μ and about 300μ. In various embodiments, the embolism protection device may be collapsed after use to facilitate removal from the vessel.

Some embodiments of the embolism protection device may be used in conjunction with a concentric wire catheter guidewire system, such as disclosed in U.S. Pat. No. 7,402,141. For instance, a system of three or more concentric wires may be used to deploy the embolism protection device in a desired location. In various embodiments, a three-wire system may be used, with an inner wire that may serve as a guidewire, a second or middle wire that may be coupled to or carry the embolism protection device, and a third or outer wire that may serve to maintain the embolism protection device in a collapsed position until it has been positioned in a desired location. In various embodiments, retraction of the third or outer wire may release the embolism protection device and allow it to expand within the lumen of the vessel. Once expanded, the embolism protection device may act as a filter to prevent embolisms from occurring.

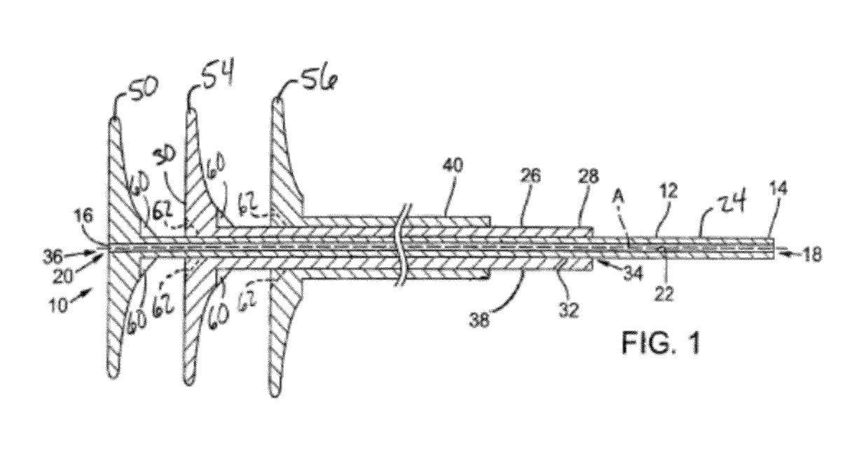

As shown in FIG. 1, an embodiment of the guidewire used to deploy the embolism protection devices disclosed herein may be a multiple concentric wire system, indicated generally at 10. In various embodiments, system 10 may include an inner wire 12 having a distal end 14 and a proximal end 16. Inner wire 12 may have a length that may be selected for a particular type of procedure to be conducted in a human blood vessel, e.g., between about 180-cm and about 300-cm. Inner wire 12 may include an opening 18 adjacent distal end 14 and an opening 20 adjacent proximal end 16, and a central lumen 22 extending between the proximal and distal openings. In various embodiments, central lumen 22 may define an inner diameter for wire 12, and wire 12 also may have a generally cylindrical outer surface 24 defining an outer diameter. Typically, the outer diameter of inner wire 12 may be between about 0.004 and 0.018 inches, and may be any size therebetween, or larger or smaller as selected for the desired procedure and for compatibility with other wires, catheters, sheaths, and other equipment. For instance, the outer diameter of inner wire 12 may be 0.010, 0.014. or 0.018 inches in specific, non-limiting examples.

Optionally, inner wire 12 may be provided with a handle 50, which may be removable adjacent proximal end 16, so that it may be used by the physician in manipulating the wire about and along a central axis A of the wire. In some embodiments, wire 12 may be constructed without transitions between sections, if it includes any sections, of the wire. Inner wire 12 also may be used in crossing a bifurcation in the vessel, and thus may be provided with a rigidity selected to allow the bifurcation crossing. In some embodiments, rigidity may be controlled by the use of braiding or the selection of various materials. For example, nitinol may be flexible, but it may become stiffer as more stainless steel is added.

A second wire 26, which may be constructed to be deployed over inner wire 12, may include a distal end 28 and a proximal end 30 and a length preferably selected to be compatible with inner wire 12. In various embodiments, a central lumen 32 of wire 26 may extend between a distal opening 34 and a proximal opening 36. As described in greater detail below, second wire 26 may couple to the embolism protection device such that the device may be advanced along inner wire 12 and selectively deployed when in a desired position.

Central lumen 32 of second wire 26 may define an inner diameter for the wire, and second wire 26 may have a generally cylindrical outer surface 38 defining an outer diameter. In various embodiments, the outer diameter of wire 26 may be between about 0.008 and 0.035 inches, for instance about 0.025 inches or about 0.035 inches in specific, non-limiting examples, and may be any size therebetween, or larger or smaller as selected for the desired procedure and for compatibility with other wires, catheters, sheaths, and other equipment.

Optionally, wire 26 may be provided with a handle 54, which may be removable, adjacent proximal end 30 that the physician may use in manipulating the wire about and along a central axis A of the wire. In some embodiments, second wire 26 may have a rigidity selected to be greater than that of inner wire 12, thus providing the system with an overall variable rigidity which may depend on the extent to which inner wire 12 extends out of second wire 26.

System 10 may also include a third or outer wire 40 having proximal and distal ends with openings and a central lumen communicating therebetween, inner and outer diameters, and a generally cylindrical outer surface as for the other wires. In some embodiments, third wire 40 may be sized to fit over second wire 26, and optionally may include a handle 56 that may be removably coupled adjacent the proximal end for manipulation of the third wire about and along central axis A. In some embodiments, third wire 40 may have a rigidity selected to be greater than the rigidity of first wire 12 and/or greater than the rigidity of second wire 26, thus providing the system with an overall variable rigidity which depends on the extent to which inner wire 12 extends out of second wire 26, and the extent to which second wire 26 extends out of third wire 40.

Third wire 40 may have an outer diameter of between about 0.010-inches and about 0.064 inches, and may be any size therebetween, or larger or smaller as selected for the desired procedure and for compatibility with other wires, catheters, sheaths, and other equipment. For instance, in specific, non-limiting examples, third wire 40 may have an outer diameter of 0.035 or 0.064 inches. Typically, the length of third wire 40 may be less than the length of second wire 26, and the length of second wire 26 may be less than that of inner wire 12.

In one specific, non-limiting example of a suitable concentric wire system, first wire 12 may have an outer diameter of about 0.014 inches, second wire 26 may have an outer diameter of about 0.025 inches, and third wire 40 may have an outer diameter of about 0.035 inches. In various embodiments, such a concentric wire system may be compatible with a 4 French catheter system. In another specific, non-limiting example, first wire 12 may have an outer diameter of about 0.018 inches, second wire 26 may have an outer diameter of about 0.035 inches, and third wire 40 may have an outer diameter of about 0.064 inches. In another specific, non-limiting example, first wire 12 may have an outer diameter of about 0.010 inches, second wire 26 may have an outer diameter of about 0.035 inches, and third wire 40 may have an outer diameter of about 0.064 inches. In various embodiments, these concentric wire systems may be compatible with a 5 or 6 French catheter system. Larger catheters also may be used, for example with an 8 French system.

In some embodiments, when the first, second, and third wires are coupled together, any of the handles of the first, second, and third wires, if present, may be used to manipulate all three wires, and also the wires may be manipulated relative to one another by simultaneous use of two or three of the handles. In some embodiments, a single handle may be used with system 10, and may be coupled to either first wire 12 or third wire 40.

In various embodiments, the length of first wire 12 may be between about 180-cm and about 300-cm, but may be other sizes as desired for particular procedures. Typically, the length of second wire 26 may be about 5-cm less than first wire 12, and the length of third wire 40 may be about 5-cm less than second wire 26.

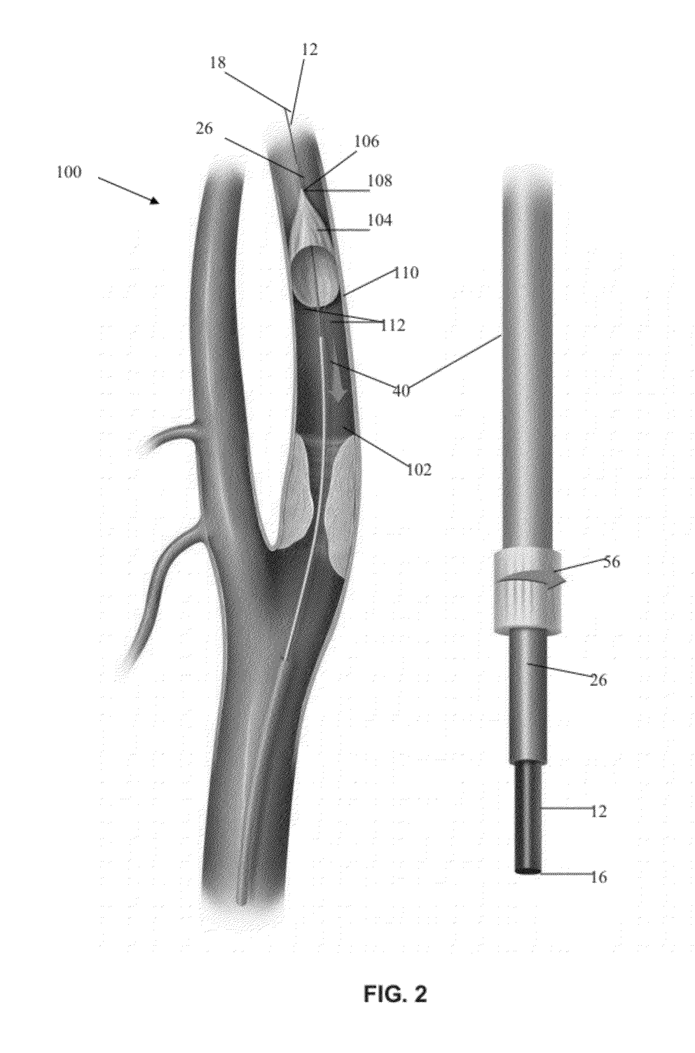

An embodiment of the invention is depicted in FIG. 2, in which an embolism protection system is indicated generally by reference number 100. System 100 includes a transporting mechanism, such as first wire 12, that is movable within a vessel 102. The vessel may be a vein, an artery, or may form part of the urinary, renal, or other fluid-transporting systems within a body. Specific, non-limiting examples of suitable arteries include the common or internal carotid artery, vertebral artery, innominate artery, or aorta. The embodiment shown in the figures relates specifically to a vein or artery having blood flowing therethrough. First wire 12 may have a tapered distal end 14, and a guide wire aperture 16 may be provided at distal end 14 to permit the catheter to be threaded upon a guide wire, as is known in the art.

Second wire 26 may be adapted to accommodate first wire 12 within central lumen 32, and third wire 40 may be adapted to correspondingly accommodate second wire 26. An embolism protection device 104 may be disposed upon wire 26 proximal to distal end 14. Although embolism protection device 104 may assume a variety of shapes in order to meet the needs of the specific application, in various embodiments it may assume a generally conical shape when in the expanded position, with second wire 26 passing through the central apex 106 of the cone near the distal end 108 of embolism protection device 104. When expanded, embolism protection device 104 may act as a nitinol mesh filter device, and proximal end 110 of embolism protection device 104 may assume a generally elliptical or circular shape against the wall of vessel 102.

In various embodiments, embolism protection device 104 may be made of a very compliant, non-tissue-traumatic metallic mesh (such as nitinol) that expands to a diameter of about 3-10 mm when fully expanded. In some embodiments, embolism protection device 104 may be a self-expanding device that is constructed so that in a compressed state the stent is biased to expand to an expanded state. When embolism protection device 104 is thus positioned and expanded, it may allow blood to pass through, thereby preventing ischemia, while also trapping and containing emboli, thus preventing tissue damage. In some embodiments, embolism protection device 104 also may have a fluted edge at proximal end 110, such that blood may pass by the exterior edge of embolism protection device 104, while the device is still adapted to retain emboli. One of skill in the art will appreciate that this may be accomplished by sizing the gaps at the fluted edge appropriately to screen out emboli while still allowing blood to flow past. In various embodiments, the inner diameter of second wire 26 may be sized such that second wire 26 fits closely over first wire 12 in order to prevent emboli from passing in the space between the concentric wires.

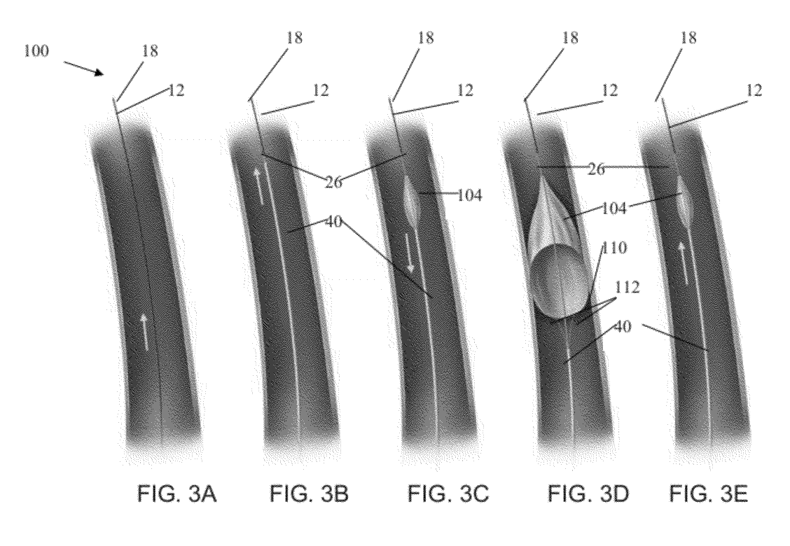

FIGS. 3A-3E depict a method of using system 10 to insert embolism protection device 104 into vessel 102. First, a user may direct first wire 12 into vessel 102 using known methods, as shown in FIG. 3A, so that distal end 14 is distal to where embolism protection device 104 will be placed. Next, the user may advance second wire 26 (which is coupled to embolic protection device 104) and third wire 40 (which maintains embolic protection device 104 in a collapsed position) together along first wire 12 to a desired location as shown in FIG. 3B. In some embodiments, first wire 12 may include a stop or trap (not shown) to ensure that second wire 26 is not advanced beyond distal end 14 of first wire 12.

Next, as shown in FIG. 3C, third wire 40 may be retracted in order to expose collapsed embolism protection device 104, which may be coupled to second wire 26. As shown in FIG. 3D, once third wire 40 is retracted, embolism protection device 104 may expand from its compressed state such that proximal end 110 may rest against or adjacent to the interior wall of vessel 102. In such an expanded position, embolism protection device 104 may allow blood to flow through its pores, yet the metallic mesh fiber captures emboli before they can cause an embolism.

Once the procedure has been completed, the embolism protection device 104 may be collapsed once again as shown in FIG. 3E but advancing third wire 40 past embolism protection device 104. As shown in FIG. 3D, embolism protection device 104 may include one or more tethers 112 to facilitate the passage of third wire 40 over embolism protection device 104, causing it to collapse. Although tethers 112 are illustrated as dual wires, they may assume any of a variety of shapes, including spirals, multiple longitudinally-aligned guide wires, or loosely-woven mesh structures. In some embodiments, any emboli trapped by embolism protection device 104 may be retained inside the device when it is collapsed. Thus, they may be removed from the vessel in a safe manner. Once embolism protection device 104 is at least partially collapsed within third wire 40, the catheter system may be withdrawn from the vessel.

In one specific, non-limiting example, the embolism protection device 104 may be used to protect the brain of a subject undergoing a percutaneous valve intervention. Prior to beginning the valve intervention, embolism protection device 104 may be advanced into a carotid artery from the right brachial artery, radial artery, or femoral artery. Such an approach is known to those of skill in the art. A 6 or 8 French sheath may be placed and, since both the innominate and the left common carotid require protection, a first wire 12 (for instance, a wire having an outer diameter of about 0.014 inches) may be passed into both internal carotid arteries. This may be accomplished with a 5 French JR4 diagnostic catheter or a V-Tech or Simmons catheter from the arm.

Once the first wires 12 are in place, (e.g., two wires, one up in the right internal carotid artery, and the other in the left internal carotid) a 0.025 inch second wire 26 and a 0.035 inch third wire 40 may be locked together and passed over first wire 12 and into both internal carotid arteries. Then, third wire 40 may be unlocked and retracted, unsheathing the embolism protection device 104 that is coupled to second wire 26. Once embolism protection device 104 is unsheathed, the device expands atraumatically into the internal carotid artery. In this position, blood may still flow through the artery via the pores in the metallic mesh of embolism protection device 104, and emboli may be trapped and collected in the device. The aortic, mitral, or pulmonic valve procedure may be safely completed, and following the procedure, third wire 40 may be passed over second wire 26 and may envelop and collapse the embolism prevention device 104 and its contents. All three wires may then be removed after the procedure on both sides.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. An intravascular embolism protection device comprising:
a first wire having a proximal end, a distal end, and an outer diameter;
a second wire having a proximal end, a distal end, an inner lumen, and an outer diameter, the inner lumen sized to accommodate the outer diameter of the first wire;
a third wire having a proximal end, a distal end, and an inner lumen, the inner lumen sized to accommodate the outer diameter of the second wire; and
a substantially conical filter device formed substantially from porous metal mesh and having a central apex, a collapsed state, and an expanded state, wherein the substantially conical filter device is adapted to couple to the second wire at or near the distal end of the second wire, the second wire passing through the central apex, and wherein the substantially conical filter device is adapted to assume the expanded state when the third wire is retracted from the distal end of the second wire, and is adapted to assume the collapsed state when the third wire is advanced over the substantially conical filter device and towards the distal end of the second wire.

2. The intravascular embolism protection device of claim 1, wherein the first, second, and third wires are formed substantially from nitinol.

3. The intravascular embolism protection device of claim 1, wherein the porous metal mesh comprises nitinol.

4. The intravascular embolism protection device of claim 1, wherein the porous metal mesh comprises pores that are sized to permit blood to pass through without allowing emboli to pass through.

5. The intravascular embolism protection device of claim 4, wherein the porous metal mesh has a pore size of from about 50 microns to about 300 microns.

6. The intravascular embolism protection device of claim 1, wherein the substantially conical filter device has a fluted edge when in the expanded state, and wherein the fluted edge creates gaps between the fluted edge and a vessel wall.

7. The intravascular embolism protection device of claim 6, wherein the gaps have a diameter of from about 50 microns to about 300 microns.

8. The intravascular embolism protection device of claim 1, wherein the central apex is at a distal end of the substantially conical filter device, and wherein the substantially conical filter device further comprises a proximal edge when in the expanded state.

9. The intravascular embolism protection device of claim 8, wherein the proximal edge is substantially circular or elliptical.

10. The intravascular embolism protection device of claim 8, wherein the proximal edge is coupled to the second wire by one or more tethers.

11. The intravascular embolism protection device of claim 10, wherein the one or more tethers comprise one or more spiral wires.

12. The intravascular embolism protection device of claim 10 wherein the one or more tethers comprise one or more longitudinal guide wires.

13. The intravascular embolism protection device of claim 10, wherein the one or more tethers comprise a loose nitinol mesh.

14. The intravascular embolism protection device of claim 10, wherein the one or more tethers are adapted to cause the substantially conical filter device to assume the collapsed state when the third wire is advanced over the second wire and toward a distal end of the second wire.

15. A method of protecting a subject from embolism during a percutaneous valve intervention, comprising:
advancing a first wire having a proximal end, a distal end, and an outer diameter through the vasculature of the subject to a carotid artery;
advancing a second wire and a third wire together along the first wire through the vasculature of the subject to the carotid artery, wherein the second and third wires have inner lumens and outer diameters, wherein the inner lumen of the second wire is sized to accommodate the outer diameter of the first wire, and wherein the inner lumen of the third wire is sized to accommodate the outer diameter of the second wire;
retracting the third wire in a proximal direction, wherein retracting the third wire exposes a substantially conical filter device coupled to the second wire at a central apex, the second wire passing through the central apex of the substantially conical filter device, wherein the substantially conical filter device is formed substantially from porous metal mesh and has a collapsed state and an expanded state, and wherein the porous metal mesh is biased to assume the expanded state.

16. The method of claim 15, wherein retracting the third wire in a proximal direction allows the substantially conical filter device to assume the expanded state.

17. The method of claim 16, wherein the substantially conical filter device assumes the expanded state atraumatically.

18. The method of claim 16, wherein allowing the substantially conical filter device to assume the expanded state comprises allowing the substantially conical filter device to substantially fill the lumen of the artery, while also permitting blood to pass through without allowing emboli to pass through the substantially conical filter device.

19. The method of claim 18, wherein in the expanded state, the porous metal mesh has a pore size of from about 50 microns to about 300 microns.

20. The method of claim 16, further comprising advancing the third wire over and collapsing the substantially conical filter device after completion of the percutaneous valve intervention.

21. The method of claim 20, wherein the substantially conical filter device comprises a substantially circular or elliptical proximal edge when in the expanded state, and wherein the proximal edge is coupled to the second wire by one or more tethers adapted to collapse the expanded substantially conical filter when the third wire is advanced in a distal direction.

22. The method of claim 15, wherein the first, second, and third wires are formed substantially from nitinol.

23. The method of claim 15, wherein the porous metal mesh comprises nitinol.
